(19) United States
(12) Patent Application Publication
(10) Pub. No.: US 2019/0388306 A1
(43) Pub. Date: Dec. 26, 2019

(54) SOLID WATER-IN-OIL COSMETIC EMULSION

(71) Applicant: CHANEL PARFUMS BEAUTE, Neuilly Sur Seine (FR)

(72) Inventors: Clémence RAVNI, Paris (FR); Hélène DE CLERMONT-GALLERANDE, Vincennes (FR); Martine YVERNOGEAU, Levallois Perret (FR); Olivia PICHOUTOU, Pantin Cedex (FR)

(73) Assignee: CHANEL PARFUMS BEAUTE, Neuilly Sur Seine (FR)

(21) Appl. No.: 16/480,429

(22) PCT Filed: Feb. 1, 2018
(86) PCT No.: PCT/FR2018/050237

§ 371 Date: Jul. 24, 2019

(30) Foreign Application Priority Data
Feb. 2, 2017 (FR) 17 50887

(51) Int. Cl. A61K 8/02; A61K 8/06; A61K 8/25; A61K 8/92; A61K 8/86; A61Q 1/06

(52) U.S. Cl. CPC A61K 8/0229; A61K 8/064; A61Q 1/06; A61K 8/92; A61K 8/86; A61K 8/25

(57) ABSTRACT

A cosmetic composition in the form of a solid water-in-oil emulsion, including: an emulsifying mixture including at least one silicone emulsifier between 0.1% and 5% and at least one emulsifying silicone elastomer between 0.05% and 3%, at least one non-volatile oil between 30% and 50% and including at least one non-volatile silicone oil and optionally a second oil chosen from at least one non-volatile ester oil, a structuring agent for the oily phase including a wax and/or an oily gelling agent, 30% to 50% of water, 0% to 2% of a volatile silicone oil and/or of a volatile hydrocarbon-based oil, and, optionally, a film-forming agent; the percentages being percentages by weight of the total weight of the composition. Also, a cosmetic method for making up and/or for nontherapeutic care of the skin or the lips, including applying to the skin or the lips the cosmetic composition.

The present invention relates to a solid water-in-oil emulsion for use in the cosmetic field, which is preferably in the form of a stick. The invention also relates to a process for making up and/or for the nontherapeutic care of the skin or lips of human beings, comprising applying said composition to the skin or lips, preferably in stick form.

Make-up and/or care compositions are commonly used to give an aesthetic appearance when applied to the lips or skin, this effect being expected to continue over time. Said compositions must especially withstand the various external factors that alter their aesthetic effect, such as saliva or sweat. In particular, cosmetics, including lipsticks, should not slide or slip in lines or wrinkles, or transfer to a surface. They must also be pleasant to apply on the lips or the skin and application should provide a feeling of comfort to the user while maintaining satisfactory aesthetic properties.

The document FR2873018, EP374332 and WO1999/47111 all describe a solid water-in-oil cosmetic emulsion applied to makeup and skin care. However these emulsions each have a sizable amount of volatile oil, which may have some disadvantages.

Volatile oils, in particular volatile silicone oils have been widely used since the year 1995 in the formulation of lipstick, in order to replace the conventional vegetable oils and liquid paraffins usually used. Indeed, the use of volatile silicone oil improves the property of “non-transferability” particularly sought for lipsticks, namely the absence of red marks on a medium that makes contact with the lips (a glass, cup, skin, clothing, etc.).

However, volatile silicone lipsticks are dull, brittle and offer quite limited comfort (including the sensation of tightness due to evaporation of volatile oils). Furthermore, evaporation of the silicones causes shrinkage of the lipstick diameter over time. In the long term, these lipsticks do not age very well because a small loss of silicone makes the lipstick even more uncomfortable and dehydrating. Furthermore, this makes it easier for the lipstick to break at the base of the holder.

The short hydrocarbon chain volatile oils, such as isododecane, isohexadecane, are also used to optimise the non-transferability of lipstick and avoid the excessive formulation incompatibilities associated with volatile silicones.

However, whatever the nature of the volatile compounds, incorporation thereof into a formula poses problems when manufacturing products. Indeed, sticks for the skin or the lips are manufactured at high temperatures to allow waxes to melt. The addition of volatile substances, whether silicone or mineral, is done at the last moment of manufacture, at a temperature as low as possible to avoid excessive evaporation of such solvents. Indeed, the evaporation of volatile solvents makes it difficult to control the final composition of the product. On the other hand, if volatile solvents have low flashpoints, particularly below the heating temperature of the “bulk” (i.e. the product in the tank) during the manufacture of the product, then security problems require the use of explosion-proof equipment to reduce the risk of manipulation.

For all these reasons, the inventors have sought a cosmetic composition for the skin or lips, especially in stick form, which includes little or no volatile oil, but which nevertheless has the usually desired properties for a stick for use of the skin or lips.

An object of the invention is to provide a makeup and/or care composition for the skin or lips, with excellent sensory properties, namely a feeling of comfort, freshness and that melts when applied to the skin or lips. In the sense used in this invention, comfort is understood to mean the absence of pulling sensation on the skin or lips.

Another object of the invention is to obtain a composition for making up and/or caring for the skin or lips, particularly in the form of sticks, which has a high water content, namely at least 30% by weight of the total weight of the composition, to enhance the feeling of freshness and comfort when applied to the skin or lips.

Another object of the invention is to provide a makeup and/or care composition for the skin or lips, which is easily spread when applied to the skin or lips with a homogeneous result once the composition is applied.

Another object of the invention is to provide a make-up stick and/or care stick for the skin or lips that has good stability over time, i.e. which does not shrink from its original form, which does not break at its base, and which does not age by desiccation or by drying out and does not show exudation (i.e. the release of oil or water on the surface).

Another object of the invention to provide a stick for the skin or lips which poses no security problem when manufacturing said stick.

Yet another object of the invention is to provide a stick for the skin or lips with good stability, appearance and sensory properties, and the manufacture of which does not require excessively high temperatures, not exceeding 95° C.

It is to the credit of the Applicant to have found that it is possible to dispense with the presence of volatile oils commonly used in cosmetic compositions, including lipstick, in the prior art.

The term “dispense with” within the meaning of the invention refers to not having any volatile oils in the compositions or else in very small quantities.

Indeed, we have found that a specific combination of specific compounds makes it advantageously possible to obtain cosmetic make up and/or care compositions stick form for the skin or lips, having all the desired properties in the scope of the invention (deposition of a matte or glossy film lack of tightness of the skin or lips, fresh feeling, a stick that will not shrink and does not age prematurely, non-constraining manufacturing, etc.).

The invention thus relates to a cosmetic composition in the form of a solid water-in-oil emulsion characterised in that it comprises:

an emulsifying mixture comprising at least one silicone emulsifier present in an amount between 0.1% and 5% and at least one elastomeric emulsifying silicone, said elastomeric silicone being present in an amount between 0.05% and 3%,

at least one non-volatile oil present in an amount between 30% and 50% and comprising at least one non-volatile silicone oil and optionally a second oil selected from at least one non-volatile ester oil,

a structuring agent of the oily phase comprising a wax and/or an oily gelling agent,

30% to 50% water,

0% to 2%, and even more preferably 0% of a volatile silicone oil and/or a volatile hydrocarbon oil, and

optionally a film-forming agent;

the percentages defined above being percentages by weight relative to the total weight of the composition.

“Solid emulsion” is understood according to the invention to be an emulsion which does not flow under its own weight when left for 1 hour at 25° C. This solid emulsion is however can flow when hot, at temperatures between 55° C. and 95° C. in order to be in the form of a stick, preferably a stick for make-up and/or for caring for the skin or the lips.

Emulsifying Silicone

The silicone emulsifier is a siloxane polymer comprising:

a fatty side chain,

an oxyethylenated or oxypropylenated side chain and/or a polyethoxylated side chain, and optionally

a silicone side chain.

The fatty side chain of the silicone emulsifier makes it possible to have good compatibility with the fatty phase of the water-in-oil emulsion.

The silicone side chain makes it possible to have good compatibility with the non-volatile silicone oil. According to one embodiment of the invention, the silicone emulsifier comprises a fatty side chain, and a silicone side chain.

More particularly according to the invention, the silicone emulsifier is selected from the group comprising:

the compound of formula (I):

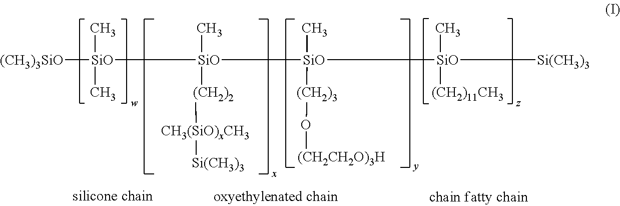

wherein w is an integer from between 1 and 1,000, x′ is an integer between 1 and 50, x, y and z representing, independently of one another, an integer between 1 and 100,

the compound of formula (II):

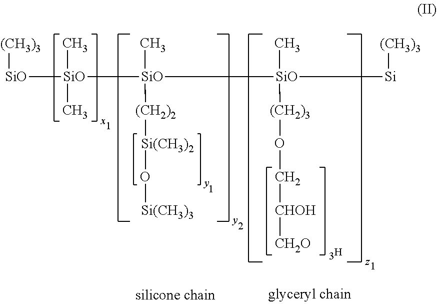

wherein x₁ is an integer between 1 and 1000, w₁ is an integer between 1 and 50, y₁ and z₁ representing, independently of one another, an integer between 1 and 100,

the compound of formula (III):

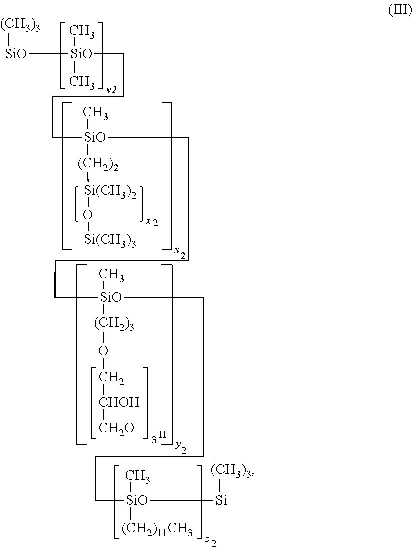

wherein w₂ is an integer between 1 and 1000, v₂ is an integer between 1 and 50, x₂, y₂ and z₂ representing, independently of one another, an integer between 1 and 100,

and mixtures thereof.

According to a particular embodiment, the silicone emulsifier is selected from the group consisting of siloxane polymers sold by the company SHIN-ETSU with reference numbers KF6038, KF6104, KF6105, KF6106, and mixtures thereof.

The compound KF6038, having the INCI name “Lauryl PEG-9 Polydimethylsiloxyethyl Dimethicone” corresponds to the general formula (I). This siloxane polymer comprises a silicone side chain, an oxyethylene side chain, and a fatty side chain (lauryl).

The compound KF6104, having the INCI name “Polyglyceryl-3 Polydimethylsiloxyethyl Dimethicone,” and the compound KF6106 having the INCI name “Polyglyceryl-3 Polydimethylsiloxyethyl Dimethicone” correspond to the general formula (II). This siloxane polymer comprises a silicone side chain and a glyceryl side chain.

The compound KF6105, having the INCI name “Lauryl Polyglyceryl-3 Polydimethylsiloxyethyl Dimethicone” corresponds to the general formula (III). This siloxane polymer comprises a silicone side chain, a glyceryl side chain and a fatty side chain (lauryl).

The silicone emulsifier is present in the cosmetic composition of the invention with a content of between 0.1% and 5%, preferably between 1% and 3%, the percentages being weight percentages relative to the total weight of the composition.

Emulsifying Silicone Elastomer

The solid water-in-oil emulsion comprises in addition to the silicone emulsifier as defined above, an emulsifying silicone elastomer, i.e. an elastomeric crosslinked organopolysiloxane, preferably with a polyhydroxylated chain.

The crosslinked organopolysiloxane elastomer can be obtained by crosslinking a diorganopolysiloxane addition reaction containing at least one silicon-bonded hydrogen and polyhydroxy or polyoxyalkylene compounds having ethylenically unsaturated groups, especially in the presence of a platinum catalyst.

More particularly, the elastomeric crosslinked organopolysiloxane is obtained by crosslinking addition reaction (A) of diorganopolysiloxane containing at least two hydrogens each bonded to a silicon and (B) glycerolated or polyoxyalkylene compounds having at least two ethylenically unsaturated groups, especially in the presence (C) of a platinum catalyst.

In particular, the organopolysiloxane may be obtained by reacting a compound of polyglycerolated dimethylvinylsiloxy terminated with methylhydrogenpolysiloxane and trimethylsiloxy end groups, in the presence of a platinum catalyst.

The compound (A) is the base reagent for the formation of elastomeric organopolysiloxane and crosslinking is effected by addition reaction of the compound (A) with the compound (B) in the presence of catalyst (C). The compound (A) is in particular an organopolysiloxane having at least 2 hydrogen atoms bonded to different silicon atoms in each molecule.

The compound (A) may have a linear chain structure, branched chain or a cyclic structure. It may have a viscosity at 25° C. of between 1 and 50,000 centistokes (cSt), particularly to have good miscibility with compound (B).

The organic groups bonded to silicon atoms of the compound (A) may be alkyl groups having 1 to 18 carbon atoms, such as methyl, ethyl, propyl, butyl, octyl, decyl, dodecyl (or lauryl), myristyl, cetyl , stearyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, a carboxylate ester group or a mercapto group.

Preferably, said organic group is selected from methyl, phenyl or lauryl. Compound (A) can be selected from trimethylsiloxy-terminated methylhydrogenpolysiloxanes, trimethylsiloxy-terminated dimethylsiloxane-methylhydrogensiloxane copolymers, dimethylsiloxane-methylhydrogensiloxane cyclic copolymers or trimethylsiloxy-terminated dimethylsiloxane-methylhydrogenosiloxane- laurylmethylsiloxane.

The compound (B) may be:

either a polyglycerolated compound corresponding to following formula (B′):

CₘH₂ₘ₋₁—O-[Gly]ₙ-CₘH₂ₘ₋₁ (B′),

wherein m is an integer between 2 and 6, n is an integer between 2 and 200, preferably between 2 and 100, preferably between 2 and 50, preferably between 2 and 20, preferably between 2 and 10, and even more preferably n is an integer between 2 and 5, and in particular n is equal to 3; Gly denotes —CH₂—CH(OH)—CH₂—O— or —CH₂—CH(CH₂OH)—O—,

or a polyoxyalkylene compound having two vinyl groups.

Advantageously, the sum of the number of ethylenic groups per molecule of the compound (B) and the number of hydrogen atoms bonded to silicon atoms per molecule of the compound (A) is at least 4.

Advantageously, the compound (A) is added in an amount such that the molar ratio between the total amount of hydrogen atoms bonded to silicon atoms in the compound (A) and the total amount of all the ethylenically unsaturated groups in the compound (B) is from 1/1 to 20/1.

The compound (C) is the catalyst for the crosslinking reaction, and is selected from chloroplatinic acid, chloroplatinic acid-olefin complexes, chloroplatinic acid-alkenylsiloxane complexes, chloroplatinic acid-diketone complexes, platinum black or platinum on a substrate.

The catalyst (C) is preferably added in an amount between 0.1 and 1000 parts by weight, preferably between 1 and 100 parts by weight, as clean platinum metal per 1,000 parts by weight of the total amount of compounds (A) and (B).

The emulsifying silicone elastomer may be conveyed in the form of a gel in at least one hydrocarbon oil and/or a silicone oil. In these gels, the emulsifying elastomer is often in the form of non-spherical particles.

Polyoxyalkylenated elastomers are especially described in patents U.S. Pat. Nos. 5,236,986, 5,412,004, 5,837,793, 5,811,487.

As polyoxyalkylenated silicone elastomer that can be used include those sold under the names “KSG-21”, “KSG-20”, “KSG-30”, “KSG-31”, KSG-32”, “KSG-33”, “KSG-210”, “KSG-310”, “KSG-320”, “KSG-330”, “KSG-340”, “X-226146” by the company Shin Etsu, “DC9010”, “DC9011” by the company Dow Corning.

The polyglycerolated elastomers are described in WO-A 2004/024798.

As polyglycerolated silicone elastomer can be used include those sold under the names “KSG-710”, “KSG-810”, “KSG-820”, “KSG-830”, “KSG-840” by the company Shin-Etsu.

The emulsifying silicone elastomer, namely crosslinked organopolysiloxane elastomer, preferably with a polyhydroxylated chain, is present in the cosmetic composition of the invention in a content between 0.05% and 3%, preferably between 0.1% and 2%, the percentages being percentages by weight relative to the total weight of the composition, it being understood that the emulsifying silicone elastomer percentages are expressed in active ingredient percentage.

According to one embodiment of the invention, the silicone emulsifier KF6038 alone or in combination with the elastomeric silicone emulsifier KSG210 is used in the cosmetic composition.

Non-Volatile Oil

Within the meaning of the present invention, the term “oil” means a liquid compound at room temperature (25° C.), and which, when introduced in a proportion of at least 1% by weight in water at 25° C., is not at all soluble in water or soluble at a level of less than 10% by weight, based on the weight of oil introduced into the water.

The term “non-volatile oil” means an oil which has a boiling temperature generally higher than 300° C. under 760 mm Hg (101325 Pa) and which has little or no vapor pressure.

Non-Volatile Silicone Oil

The term “silicone oil” or “oil of silicone” means an oil comprising at least one silicon atom, and in particular at least one Si—O group.

More particularly, according to one embodiment of the invention, the non-volatile silicone oil is chosen from the group comprising polydimethylsiloxane (PDMS) (dimethicones: INCI name) containing at least 8 silicon atoms, polyalkylmethylsiloxanes the alkyl chain of which contains from 8 to 20 carbon atoms, alkyl dimethicones, vinyl methyl methicones, oils identified by the INCI name phenyltrimethicones and mixtures thereof.

According to one embodiment of the invention, the non-volatile silicone oil has a viscosity at 25° C. of greater than or equal to 5 cSt. Preferably the non-volatile silicone oil has a viscosity at 25° C. less than or equal to 10 cSt.

As an example of the dimethicone, “dimethicone 10 cSt” may be mentioned, which is marketed by:

SHIN-ETSU under reference KF-96-A, especially KF-96-A-10CS,

Wacker Chemie under reference Belsil® DM10

Dow Corning under reference Xiameter-PM-200 SIL FLUID 10CS,

Evonik Industries AG under the reference Abil 10.

As an example of the dimethicone “dimethicone 5 cSt” may be mentioned, marketed by Dow Corning under the reference Xiameter PMX-200 SIL FLUID 5CS.

The non-volatile silicone oil of the invention can be used alone or mixed with other non-volatile oils.

The non-volatile oil that may especially be used in the composition of the invention in a mixture with the non-volatile silicone oil, is a non-volatile ester oil.

A non-volatile “ester oil” is understood to be an oil comprising at least one ester functional group in its molecule. Advantageously, the non-volatile ester oil is a monoester.

The non-volatile ester oil may be chosen from the monoesters of formula R₁COOR₂ wherein R₁ represents a linear or branched hydrocarbon chain having from 4 to 40 carbon atoms, preferably from 4 to 30 carbon atoms, and preferably 7 to 20 carbon atoms, and R2 represents a branched hydrocarbon chain containing from 3 to 40 carbon atoms, preferably 10 to 30 carbon atoms, and preferably from 16 to 26 carbon atoms.

As an example of a non-volatile ester oil, there may be mentioned more preferably palmitate, 2-ethylhexyl, isopropyl palmitate, isooctyl palmitate, isocetyl palmitate, isodecyl palmitate isostearyl palmitate, 2-octyl-decyl, isodecyl neopentanoate, isocetyl octanoate, isononyl isononanoate, isodecyl isononanoate, tridecyl isononanoate, laurate hexyl laurate, 2-hexyl-decyl, isopropyl myristate, isocetyl myristate, isotridecyl myristate, 2-octyldodecyl myristate, isopropyl isostearate, octyldodecyl stearate, 2-octyldodecyl erucate, isostearyl isostearate, erucate 2-octyldodecyl, diisostearyl malate, and mixtures thereof.

Depending on the intended uses, other non-volatile oils may be added to the compositions of the invention.

In the context of the present invention, the term “oil” also includes lipophilic fats that are capable of undergoing a reversible liquid/solid change and have an anisotropic crystalline organisation in the solid state, but are different from waxes by the fact they contain, at a temperature of 23° C., a liquid fraction and a solid fraction. A compound of this type is in particular a mixture of sterol esters such that the mixture of cholesterol and lanosterol ester available from the manufacturer Croda under the trade name Super Sterol Ester®.

The oils that can be used in the composition according to the invention can be chosen from the group comprising hydrocarbon oils; (poly) esters and synthetic (poly) ethers, in particular C6-C20 acid (poly) esters and C6-C20 alcohols, preferably branched such as isononyl isononanoate; vegetable oils; branched and/or unsaturated fatty acids; branched fatty alcohols and/or unsaturated such as octyldodecanol; fluorosilicone oils; fluorinated oils; and mixtures thereof.

In the event that obtaining a gloss composition is desired, for example an eyeshadow, a balm lip gloss or glossy lipstick, it is preferable to use in the present invention at least one glossy oil, i.e. an oil with a refractive index greater than 1.45, preferably greater than 1.47.

Examples of glossy oils include hydrocarbon oils such as squalane, phytosqualane, polybutene, hydrogenated polyisobutene, hydrogenated polydecene; the phenylated silicone oils such as those identified:

by the INCI name “phenyl trimethicone,” especially that marketed by Rhodia under the name MIRASIL® PTM

by the INCI name “phenylpropyldimethylsiloxysilicate”, in particular that marketed by General Electric under the name Silshine® 151

by the INCI name “trimethylpentaphenyl trisiloxane”, in particular that marketed by Dow Corning under the trade name DC PH® 1555 HRI.

We may also mention, as glossy oils:

fluorinated silicones identified by the INCI name “dimethicone perfluorononyl”, in particular that marketed by Phoenix Chemical, Inc. under the trade name Pecosil FS (FSU, FSL, etc.) and marketed by Biosil Technologies under the trademark Biosil Basics® (Fluorosil LF, 14, and the like);

amino ceramide identified by the INCI name “phytosteryl/octyldodecyl lauroyl glutamate”, in particular that marketed by Ajinomoto under the trade name Eldew® PS203.

Other examples of glossy oils are natural oils, especially castor seed oil, jojoba oil, limnanthes oil; branched fatty alcohols and/or unsaturated such as octyldodecanol, mono- and polyesters of fatty acids and/or fatty alcohols having a fatty chain comprising from 6 to 20 carbon atoms, in particular mono- and hydroxy polyesters and fatty alcohols, such as diisostearyl malate, benzoic acid esters and fatty alcohols, such as alkyl benzoates C12-C15, the polyol polyesters, particularly (di)pentaerythritol, such as pentaerythrityl tetraisostearate, dipentaerythrityl pentaisononanoate and dipentaerythrityl C5-C9 esters, polyglycerol esters such as those identified by the INCI name “bis-diglyceryl polyacyladipate-1” marketed by Sasol under the tradename Softisan® 645, or polyglyceryl-2 triisostearate marketed under the tradename Salacos 43N by Nisshin OilliO Group Ltd., trimethylolpropane esters, such as triethylhexanoate trimethacrylate, marketed in particular by Kokyu Alcohol Kogyo under the tradename Kak® TTO, propylene glycol esters, such as propylene glycol dibenzoate, marketed in particular by Inolex under the trade name Lexfeel Shine®, isocetyl stearoyl stearate; hydrogenated castor oil polyesters, such as esters marketed by Kokyu Alcohol Kogyo under the trade names Risocast® DA-H and Risocast® DA-L.

In cases where it is desired to obtain a matte composition, such as an anti-dark eye circle product, a foundation or a matte lipstick, the oils used in the composition of the invention are non-glossy oils.

Examples of appropriate non-glossy oils in the cosmetic compositions of the invention include (poly) ester and (poly) synthetic ethers, in particular C6-C20 acid (poly) esters and C6-C20 alcohols, preferably branched, such as isononyl isononanoate and isostearyl neopentanoate; di (alkyl C6-C20) carbonates such as dicaprylyl carbonate marketed by BASF under the name Cetiol CC; branched and/or unsaturated fatty acids; polyol polyesters, particularly (di) pentaerythritol such as pentaerythritol tetraoctanoate, silicone oils such as linear polydimethylsiloxanes of viscosity greater than or equal to 5 cSt; and mixtures thereof.

According to one embodiment, the compositions of the invention comprise a mixture of the above-mentioned oils.

According to an advantageous embodiment of the invention, the non-volatile oil, used in combination with the non-volatile silicone oil is chosen from the group comprising brilliant non-volatile oils and in particular mono- and acid polyesters fatty and/or fatty alcohols having a fatty chain comprising from 6 to 20 carbon atoms, in particular mono- and polyesters of hydroxy acids and fatty alcohols, such as diisostearyl malate.

According to another advantageous embodiment of the invention, the non-volatile oil, used in combination with the non-volatile silicone oil is chosen from the group comprising non-glossy non-volatile silicone oils and especially linear polydimethylsiloxanes of a viscosity greater than or equal to 5 cSt.

As an example of the invention, the non-volatile silicone oil used in the composition is associated with a non-volatile ester oil, in particular 2-ethylhexyl palmitate and/or diisostearyl malate.

The non-volatile oil is present in the cosmetic composition of the invention in a content between 30% and 50%, the percentages being percentages by weight relative to the total weight of the composition.

non-volatile silicone oil is present in the cosmetic composition of the invention in an amount between 10% and 50%, the percentages being percentages by weight relative to the total weight of the composition.

In a particularly interesting embodiment of the invention, the composition comprises:
an emulsifying mixture comprising at least one silicone emulsifier present in an amount between 0.1% and 5% and at least one elastomeric silicone emulsifying, said elastomeric silicone being present in an amount between 0.05% and 3%,
at least one non-volatile oil present in an amount between 30% and 50% and comprising at least one non-volatile silicone oil in an amount between 10% and 50% and a second oil selected from at least one non-volatile ester oil,
2% to 12%, preferably 6% to 10%, of a structuring agent for the oily phase,
30% to 50% water,
0% to 2% and preferably 0% of a volatile silicone oil and/or a volatile hydrocarbon oil, and
optionally a film-forming in an amount less than or equal to 10%;

the percentages defined hereinbefore being percentages by weight relative to the total weight of the composition.

According to a preferred embodiment of the invention the composition comprises a film-forming agent.

The film-forming agent is chosen in particular from silicone resins. Among these include particularly trimethylsiloxysilicates resins such as that marketed by Wacker Chemie AG under the name Belsil® TMS 803.

The film-forming agent can also be selected from silicone acrylate polymers. Among them the Acrylates/Polytrimethylsiloxymetliaciylate Copolymer include for example such as that marketed by Dow Corning under the name Dow Corning® FA 4002 ID Silicone Acrylate.

The film-forming agent may also be selected from polybutene polymers such as that marketed by Rossow under the name Parleam® HV.

The film-forming agent may also be selected from vegetable resins such as Shorea Robusta resin such as pre-dispersed in octyldodecanol marketed by the company Kahlwax under the name Kahlresin® 6723.

The film-forming agent can also be selected from a mixture of the film-forming agents mentioned above.

According to a particularly advantageous embodiment, the composition of the invention comprises, as a film-forming agent a mixture of Belsil® TMS 803 and Kahlresin® 6723.

The film-forming agent, when it is not optional, is present in the composition of the invention in an amount between 1% and 15%, preferably between 1% and 10%, the percentages being percentages by weight relative to the total weight of the composition, provided that when the film-forming agents are pre-dispersed in a solvent the percentage corresponds to the percentage of active material.

According to a particularly advantageous embodiment, the composition of the invention comprises, as a film-forming agent, a mixture of 7% of Belsil TMS 803 and 2% of Kahlresin® 6723, the percentages being percentages by weight of the total weight of the composition.

Structuring Agent of the Oily Phase

The structuring agent of the oily phase comprises at least one wax and/or at least one oily gelling agent.

The term “wax” means a fatty material with a reversible liquid /solid change having a melting point greater than 30° C. and usually below 110° C. which is liquid under the conditions of preparation of the composition and has an anisotropic crystalline organisation in the solid state.

The term “oily gelling agent” refers to a substance capable of solidifying or gelatinizing the oil introduced into the composition of the invention.

According to one embodiment of the invention, the structuring agent is a mixture of at least one wax and at least one oily gelling agent.

According to another embodiment of the invention, the structuring agent is a mixture of at least two waxes.

The appropriate wax to the cosmetic compositions of the invention comprises at least one polar wax and/or at least one apolar wax.

Polar wax means a wax comprising at least one heteroatom such as oxygen, nitrogen, silicon or phosphorus.

In particular, the polar wax may be selected from the group consisting of beeswax, carnauba wax, candelilla wax, cotton wax, rice bran wax, berry wax, beeswax, Chinese insect wax, montan wax, lanolin and the alcohols derived therefrom, acetyls, esters, polyethoxylates, kapok wax, sugarcane wax, hexyl laurate, jojoba wax, shellac wax, cholesterol polyethoxylated ether, synthetic beeswaxes marketed by Koster Keunen under the trade designation Kester Wax K82H, and mixtures thereof.

We may also mention waxes of vegetable esters chosen from the group comprising the mixture of jojoba esters, polyglycerin-3, Acacia decurrens flower wax and sunflower seed wax, said mixture being commercially available from Gattefosse under the trade name Acticire®, jojoba esters marketed by Floratech under the tradename Floraesters 60 or Floraesters 70, hydrogenated alkyl esters or alkyl esters marketed by Sophim under the trade name Photowax, as well as oleate esters of hydrogenated lauroyl marketed under the name Phytowax Olive 12L44.

Non-polar wax means a hydrocarbon wax and/or a silicone wax.

The term “apolar hydrocarbon wax”, a wax containing only carbon and hydrogen atoms and containing no hetero atoms such as oxygen, nitrogen, silicon or phosphorus.

Examples of suitable nonpolar hydrocarbon waxes in the compositions of the invention include polyethylene wax marketed by New Phase Technologies under the name Performalene® 400 (P400) or by Jeen International Corporation under the name Jeenate 3H, a mixture of high molecular weight linear polyethylene and of ethylene/propylene copolymer marketed by SAFIC-ALCAN under the name Lipwax® PZ80-20, synthetic wax marketed by Sasol under the name Sasol wax C80, synthetic waxes and vegetable waxes, such as a mixture of synthetic wax and carnauba wax (Copernica cerifera) commercially available from Strahl & Pitsch under the name Smart wax 202, a mixture of synthetic wax, candelilla wax and carnauba wax (Copernica cerifera) commercially available from Strahl & Pitsch under the name Smartwax 7743S, Fischer Tropsch waxes marketed by Cirebelle under the name Cirebelle 303, and mixtures thereof.

The term “silicone apolar wax” means a wax comprising a silicon heteroatom.

Examples of suitable silicon apolar waxes in the compositions of the invention include the C20-24 alkyl dimethicone marketed by Siltech under the name Silwax D2024, the C24-28 alkyl dimethicone marketed by Evonik Industries AG under the trademark Abil Wax and mixtures thereof.

According to one embodiment of the invention, the wax is a nonpolar wax or a mixture of non-polar waxes, said non-polar wax is a hydrocarbon apolar wax or a mixture of apolar hydrocarbon waxes.

More particularly, according to still another embodiment of the invention the wax is selected from the group consisting of polyethylene wax, alone or in combination with another polyethylene wax, a polyethylene wax and wax mixture of plant esters, a mixture of synthetic wax and carnauba wax, jojoba esters, and mixtures thereof.

Some examples of wax that may be mentioned are polyethylene wax “Jeenate 3H”, polyethylene wax “Jeenate 3H” and polyethylene wax “Performalene 400” blend, a polyethylene wax “Jeenate 3H” and wax “Smart wax 202” blend, or a blend of polyethylene wax “Jeenate 3H” and jojoba esters “Floraester 70”.

According to one embodiment of the invention, the wax is present in the cosmetic composition of the invention in a content of between 2% and 12%, preferably between 6% and 10%, the percentages being percentages by weight relative to the total weight of the composition.

The wax and the quantity thereof, are chosen so as to obtain a cosmetic composition having an acceptable hardness, good stability, good sensory properties, while maintaining the “bulk” heating temperature as low as possible during the process of manufacture of said composition.

The oily gelling agent that can be included in the cosmetic composition of the invention is selected from the group comprising a silicone polymer; a vinyl dimethicone/dimethicone cyclic copolymer; a polyamide resin or a resin poly (ester-amide); a copolymer of styrene and at least one olefin other than styrene; N-acyl glutamic acid diamide; a dextrin ester; a sucrose ester and mixtures thereof.

As an example of a silicone polymer suitable as oily gelling agent, there may be mentioned a silicone polymer obtained by self-polymerisation in the presence of a catalyst of an organopolysiloxane functionalised with epoxy and hydrosilylated groups, marketed by General Electric under the name Velvesil® 125.

As an example of a cyclic copolymer of vinyl dimethicone/dimethicone suitable as oily gelling agent, there may be mentioned the agent marketed by Jeen under the tradename Jeesilc® PS (including PS VH, PS VHLV, PS CM, PS CMLV and PS DM).

By way of examples of resins of polyamide resins or poly (ester-amide) as appropriate oily gelling agents, there may be mentioned ester-terminated polyamides (ETPA), ester-terminated poly (ester-amides) (ETPEA), tertiary amide-terminated polyamides (ATPA), polyalkyleneoxy terminated polyamides (PAOPA) or polyether polyamides (PEPA).

Examples of ester-terminated polyamides (ETPA) are those identified by the INCI name “Ethylenediamine/Stearyl Dimer Dilinoleate Copolymer” and available, for example, under the Arizona Chemical trade name Uniclear® 100VG.

Examples of ester terminated poly (ester-amides) (ETPEA) are those identified by the INCI name polyamide-8, which are “ethylenediamine bis-stearyl ethylenediamine/neopentyl glycol/stearyl dimer dibenzoate copolymers” and available, e.g., under the Croda trade name Oleocraft® LP-20 PA-MV.

Examples of tertiary amide-terminated polyamides (ATPA) are those identified by the INCI name “Ethylenediamine/of Bis-Di-C14-18 hydrogenated dimer Alkyl Amide diilinoate copolymer” and available, for example, under the Arizona Chemical trade name Sylvaclear® A200V or Sylvaclear® A2614V or those identified by the INCI name “diisostearyl malate and bis-dioctadecylamide dimer dilinoleic acid/ethylene” and available, for example, under the Kokyu Alcohol Kogyo trade name Haimalate PAM.

Examples of polyalkyleneoxy terminated polyamides (PAOPA) are those identified by the INCI name Polyamide-3 and available, for example, under the name Sylvaclear® AF1900V, Sylvaclear® PE1800V and Sylvaclear® PA1200V by Arizona Chemical.

Examples of polyether polyamides (PEPA) are those identified by the INCI name Polyamide-6 and available, for example, under the name of Sylvaclear® PE400V by Arizona Chemical.

By way of examples copolymers of styrene and at least one olefin other than styrene, such as an appropriate oily gelling agent, there may be mentioned copolymers, in particular block copolymers, of styrene and at least one olefin other than styrene selected from ethylene, propylene, butylene, butadiene, isoprene and mixtures thereof. These copolymers are marketed by Shell under the trade name Kraton® and may in particular be a styrene-ethylene-propylene copolymer, styrene-ethylene-butylene, styrene-butadiene, styrene-isoprene, styrene-butadiene-styrene, styrene-isoprene-styrene or a styrene-ethylene-butylene-styrene copolymer or an ethylene/propylene/styrene or butylene/ethylene/styrene or any mixture of the aforementioned copolymers, for example a mixture of ethylene/propylene/styrene copolymer and butylene/ethylene/styrene. An example of a mixture of copolymers used according to the invention consists of Dekagel® products marketed by Jan Dekker and in particular Dekagel® HV2004. Other examples of styrene copolymers used in the present invention are those marketed by Penreco under the trade names Versagel® M, ME, ML, MP, MC, MD and MN including Versagel® ME, and especially Versagel® ME 2000.

By way of examples of N-acyl glutamic acid diamides suitable as an oily gelling agent, there can especially be mentioned diamide of an N-acyl glutamic acid having a straight chain alkyl group such as dibutyl lauroyl glutamide and an N-acyl glutamic acid diamide having a branched chain alkyl group, such as dibutyl ethylhexanoyl glutamide. Dibutyl lauroyl glutamide is commercially available as GP-1 and dibutyl ethylhexanoyl glutamide is commercially available under the name EB-21, and are both marketed by Ajinomoto.

By way of examples of suitable dextrin esters as an oily gelling agent, there may be mentioned fatty acids dextrin esters, such as dextrin palmitate.

By way of examples of suitable sucrose esters as an oily gelling agent, there may be mentioned fatty acid sucrose esters such as sucrose tetrastearate triacetate available under the Sisterna trade name Sisterna® A10E-C.

According to an advantageous embodiment of the invention, there may be mentioned more particularly as an oily gelling agent, sucrose esters such as sucrose tetrastearate triacetate, marketed by Sisterna under the tradename Sisterna® A10E-C.

According to one embodiment of the invention, the gelling agent of the oily phase is present in the cosmetic composition of the invention in an amount between 2% and 12%, preferably between 6% and 10%, the percentages being weight percentages based on the total weight of the composition.

The structuring agent of the oily phase is present in the cosmetic composition of the invention in an amount between 2% and 12%, preferably between 6% and 10%, the percentages being weight percentages based on the weight total composition.

The composition of the invention is more particularly characterised in that it comprises:

an emulsifying mixture comprising at least one silicone emulsifier present in an amount between 0.1% and 5% and at least one silicone elastomer, said elastomer silicone emulsifier being present in an amount between 0.05% and 3%

at least one non-volatile oil present in an amount between 30% and 50% and comprising at least one non-volatile silicone oil and optionally a second oil selected from at least one non-volatile ester oil,

2% to 12%, preferably 6% to 10%, of a structuring agent of the oily phase as defined above,

the percentages defined above as being percentages by weight relative to the total weight of the composition.

Water

According to an advantageous embodiment, the cosmetic composition of the invention comprises water, preferably deionised water, in an amount between 30% and 50%, preferably between 35% and 45% and more preferably in an amount of about 40%, the percentages being percentages by weight relative to the total weight of the composition.

Volatile Oil

According to an advantageous embodiment of the invention, the composition contains no (0%) or very little (maximum 2% by weight relative to the total weight of the composition) of volatile oil.

The term “volatile oil” means an oil capable of evaporating on contact with the skin in less than one hour at room temperature and atmospheric pressure.

The volatile oil is a volatile cosmetic oil that is liquid at room temperature, especially having a non-zero vapor pressure at ambient temperature and atmospheric pressure, in particular having a vapor pressure ranging from 0.13 Pa to 40,000 Pa (0.001 to 300 mmHg), preferably between 1.3 Pa and 13,000 Pa (0.01 to 100 mmHg), and even more preferably between 1.3 Pa and 1300 Pa (0.01 to 1000 mm Hg).

Volatile oils include volatile silicone oils and/or volatile hydrocarbon oils.

Volatile silicone oils optionally used in the compositions of the invention are linear or cyclic, have from 2 to 7 silicon atoms, optionally alkyl or alkoxy groups having 1 to 10 carbon atoms, and have a viscosity, at ambient temperature, of less than 5 cSt.

By way of examples of volatile silicone oil, there can be mentioned more particularly hexamethylcyclotrisiloxane, octamethylcyclotetrasiloxane, decamethylcyclopentasiloxane, dodecamethylcyclohexasiloxane, cyclotetradimethylsiloxane, cyclopentadimethylsiloxane, cyclohexadimethylsiloxane, hexamethyldisiloxane, octamethyltrisiloxane, hexylheptamethyltrisiloxane, octylheptamethyltrisiloxane, decamethyltetrasiloxane, dodecamethylpentasiloxane, heptamethylhexyl trisiloxane, heptamethyloctyl trisiloxane and mixtures thereof.

Regarding the volatile hydrocarbon oil there can be mentioned more particularly a short-chain hydrocarbon oil, volatile linear alkanes such as for example described in the document FR2933865 incorporated by reference.

By way of examples of short chain hydrocarbon oil(s), there can be in particular one(s) chosen from the group consisting of isododecane, isodecane, isohexadecane, and mixtures thereof.

By way of examples of volatile linear alkanes, there may be mentioned those having hydrocarbon chains such as the following:

C9-C17, C10-C14, such as a mixture of undecane and tridecane, marketed by BASF Care Creations under the name Cetiol® Ultimate,

C15-19, such as those marketed by Seppic under the name Emogreen L15,

Cl 12-14, such as those marketed by Biosynthis under the name Vegelight 1214LC.

According to an advantageous embodiment of the invention, the composition as defined above may further comprise a non-silicone emulsifier, a humectant, a filler, a colorant, a preservative and/or sodium chloride.

Non-Silicone Emulsifier

The non-silicone emulsifier is chosen especially from the group comprising polymers of the type glycol fatty acid polyoxyalkylene ester, such as Polysorbate 60 marketed by Croda under the name Tween 60, Polysorbate 20 marketed by Croda under the name Tween 20, Polysorbate 85 marketed by Seppic under the name Montanox 85, and mixtures thereof, and preferably the emulsifier identified by the INCI name Polysorbate 60.

The cosmetic composition of the invention comprises from 0% to 5% of a non-silicone emulsifier, preferably from 0.1% to 1%, the percentages being percentages by weight relative to the total weight of the composition.

Humectant

The term humectant is understood to mean a compound comprising at least two hydrophilic groups, that is to say groups which form hydrogen bonds with water molecules, such as hydroxyl, amino and/or carboxyl.

A suitable humectant in the cosmetic composition of the invention is especially selected from the group consisting of glycerol; glycols, such as propylene glycol, butylene glycol and hexylene glycol; sugar alcohols or sugar polyols such as sorbitol, xylitol and maltitol; polymer polyols such as polydextrose; alpha hydroxy acids such as lactic acid; hyaluronic acid; urea; L-pyrrolidone carboxylic acid (PCA); and mixtures thereof.

According to an advantageous embodiment of the invention, the humectant is glycerin and/or glycols.

The cosmetic composition of the invention comprises from 0% to 10% of humectant, preferably from 1% to 6% humectant, the percentages being percentages by weight relative to the total weight of the composition.

Additives

The term additives is understood to mean particles that are colourless or white, mineral or synthetic, solids of any shape, which are insoluble and dispersed in the medium of the composition irrespective of the temperature at which the composition is manufactured. The additives can be inorganic or organic and of any form, platelet-shaped, spherical or oblong, irrespective of the crystallographic form (for example lamellar, cubic, hexagonal, orthorhombic, etc.). The fillers may be formed of several layers of chemical and/or different physical forms and can be platelets coated with spherical additives. They can be modified by using different surface treatments.

The additives that can be used in the cosmetic compositions of the invention are selected from the group consisting of talc, mica, silica, kaolin, boron nitride, lauroyl lysine, sericite, cellulose beads, glass beads, starch, starch modified by octenyl succinic anhydride, silicone resins such as polymethylsilsesquioxane available under the Momentive Performance Materials tradename Tospearl 2000B, derived silicone elastomers powders such as elastomer silicone coated with a silicone resin available under the ShinEtsu trade name KSP100 or KSP300, and mixtures thereof.

The cosmetic composition of the invention comprises from 0% to 25% of additives, preferably from 2% to 10%, the percentages being percentages by weight relative to the total weight of the composition.

The additives are advantageously in powder form.

Colorants

The composition may also comprise at least one colorant selected from the group comprising water-soluble dyes, liposoluble dyes, particles having the effect of colouring and/or opacifying the composition, such as pigments, nacres, lacquers (water-soluble dyes adsorbed on an inert inorganic support), and mixtures thereof.

These colorants may optionally be surface-treated with a hydrophobic agent such as silanes, silicones, fatty acid soaps, fluoroalkyl phosphates C9-C15, acrylate/dimethicone, mixed copolymers fluoroalkyl phosphate C9-C15/silicone, lecithins, carnauba waxes, polyethylene, chitosan and optionally acylated amino acids, such as lysine lauroyl, stearoyl disodium glutamate and aluminium acyl glutamate.

The pigments may be inorganic or organic and natural or synthetic.

Examples of inorganic pigments include iron oxides, titanium or zinc, ultramarine blue, Prussian blue, manganese violet, chromium oxide, and composite pigments, and goniochromatic, pearlescent, interfering, photochromic or thermochromic pigments, with this list not being limiting.

The organic pigments that can be used in the invention include Red 7, Red 6, Yellow 5, Yellow 6, Blue 1, Red 22, Red 28, Red 30, Red 33, Red 36, carbon black, lacquers made from cochineal carmine, barium, strontium, calcium, aluminium or the diketopyrrolopyrroles (DPP) described in EP-A-542669, EP-A-787 730, EP-787731 and WO-A-96/08537.

When it comprises one or more pigments, the cosmetic composition according to the invention further comprises a dispersing agent. By way of examples of dispersant diisostearyl malate may be mentioned, which is also an ester oil.

Pearlescent agents may be chosen from those conventionally present in makeup products, such as based on mica/titanium dioxide products. They may also be products made from mica/silica/titanium dioxide, made from synthetic fluoro/titanium dioxide (Sunshine® of Maprecos), made from calcium sodium borosilicate/titanium dioxide (Reflecks® Engelhard) or made from calcium borosilicate and aluminium/silica/titanium dioxide (Merck Ronastar®).

The cosmetic composition of the invention comprises from 0% to 10% colorant, preferably from 2% to 6%, the percentages being percentages by weight relative to the total weight of the composition.

As additives, colorants are advantageously in powder form.

Preservatives

The cosmetic composition may also comprise a preservative agent, in particular selected from the group consisting of phenoxyethanol, potassium sorbate, sodium dehydroacetate, chlorphenesin, parabens such as methylparaben or propylparaben, and mixtures thereof.

The cosmetic composition of the invention comprises from 0% to 2% of a preservative, preferably 0.1% to 1%, the percentages being percentages by weight relative to the total weight of the composition.

Sodium Chloride

The cosmetic composition may also comprise sodium chloride in order to further stabilise the latter. Sodium chloride would be present in an amount between 0% and 1.5%, preferably between 0.3% 0.7% by weight relative to the total weight of the composition.

According to an advantageous embodiment of the invention, the composition as defined above is more particularly characterised in that it comprises:

0% to 5% of a non-silicone emulsifier such as defined above, preferably 0.1% to 1%

0% to 10% of the humectant as defined above, preferably 1% to 6%,

0% to 25% of an additive such as defined above, preferably 2% to 10%,

0% to 10% of a colorant as defined above, preferably 2% to 6%,

0% to 2% of a preservative as defined above, preferably 0.1% to 1%,

0% in 1.5% sodium chloride, preferably 0.3 to 0.7%,

the percentages defined above being percentages by weight relative to the total weight of the composition.

Cosmetic Active Agent

The composition according to the invention may further include a cosmetic active agent, especially selected from the group consisting of moisturising agents such as hyaluronic acid, L-pyrrolidone carboxylic acid (PCA); healing agents such as allantoin; anti-aging agents such as anti-oxidants, such as ascorbic acid and/or its alkyl or phosphoryl esters, tocopherol and/or its esters; and mixtures thereof.

Sequestering Agent

The composition of the invention may also comprise a sequestering agent, such as salts of ethylenediamine tetraacetic acid edetic acid (EDTA).

UV Filter

The composition of the invention may further comprise a UV filter selected from the group consisting of organic sunscreen agents, inorganic filters and mixtures thereof.

These include, by way of organic filters dibenzoylmethane derivatives (including butyl methoxydibenzoylmethane), derivatives of cinnamic acid (including ethylhexyl methoxycinnamate), salicylates, para-aminobenzoic acids, β,β-diphenylacrylates, benzophenones, benzylidenecamphore derivatives, phenylbenzimidazoles, triazines, phenylbenzotriazoles and/or anthranilic derivatives.

There may be mentioned as inorganic filters, filters made of inorganic oxides in the form of pigments or nanopigments which can be uncoated or non-coated, in particular made from titanium dioxide or zinc oxide.

Sweetening Agent

When the composition of the invention is in the form of a lip product, it may further comprise a sweetening agent, in particular selected from the group consisting of sorbitol, sucrose, xylitol, acesulfame-K, sodium saccharinate and mixtures thereof.

When the cosmetic composition of the invention is in the form of an anti-dark eye circle product, it may further comprise an astringent agent (which reduces the appearance of pores) and/or a bleaching agent.

Examples of additives that may be added in the cosmetic compositions of the invention are described in the CTFA dictionary (International Cosmetic Ingredient Dictionary) and in the Handbook published by “The Cosmetic, Toiletry and Fragrance Association, 9th Edition, 2002”.

Of course, someone skilled in the art may select these possible additional common cosmetic ingredients, and/or their quantity, such that the advantageous properties of the composition according to the invention are not, or not substantially impaired by said addition.

The composition of the invention is in the form of a stable solid water-in-oil emulsion with a homogeneous appearance.

The term stable is understood to mean an emulsion which does not release water or oil and which remains in its original form, even exposed to ovens at a temperature between 5° C. and 40° C. for 3 months.

The term homogeneous appearance is understood to mean an emulsion with a surface that is smooth and uniform to the naked eye.

The cosmetic composition of the invention as defined above may be further characterised in that it has a hardness of between 70 g and 150 g (grams), preferably between 80 g and 120 g, and even more preferably between 80 g and 100 g.

The hardness of the composition, which is expressed in grams (g) is determined by measuring the compression strength measured at 20° C. using a texturometer marketed by Swantech society under the name “TA-XT Plus Microstable System”. The texture analyser is equipped with a stainless-steel cylinder with a diameter of 2 mm moving at the speed of 1 mm/s and penetrating into the composition at a depth of 3 mm.

The value of the hardness is the compressive strength measured divided by the area of the texturometer cylinder in contact with the composition. The samples were hot cast and filled to the brim in a round petri dish of 60 mm radius size and 15 mm in height. The samples thus prepared were stored from 24 hours to 48 hours at 20° C. prior to measurement.

The composition according to the invention can advantageously be used for non-therapeutic care and/or make up of the skin or lips.

According to this embodiment, the invention relates to a cosmetic composition in the form of a solid water-in-oil emulsion as defined above, characterised in that it is in the form of a stick, preferably a makeup stick for the skin or lips and/or a non-therapeutic care stick for the skin or lips.

The invention also relates to a cosmetic process for making up and/or for the nontherapeutic care of the skin or the lips, characterised in that it consists in applying a composition as defined above to the skin or the lips.

The composition according to the invention can be prepared according to the following procedure.

Firstly, the mixture of constituents of the oily phase is prepared by mixing and heating to a temperature between 70° C. and 120° C., the silicone emulsifier system (i.e. the silicone emulsifier and optionally the emulsifying silicone elastomer), non-volatile silicone oil and structuring agent of the oily phase.

The mixture was stirred and maintained at a temperature between 70° C. and 95° C.

Optional ingredients such as other non-volatile oils, colorants etc. can be added to the oil phase.

Secondly, the aqueous phase is prepared, mainly comprising water, and optionally a non-silicone emulsifier, and preservatives.

The aqueous phase is then heated to a temperature between 70° C. and 95° C., then the aqueous phase is added in the oil phase and is stirred using a turbine until the water-in-oil emulsion is obtained.

The emulsion is then cast at a temperature between 55° C. and 95° C. in a container suitable to give it the shape of a stick, then is cooled to room temperature until a solid emulsion in obtained.

The following examples are given for illustrative purposes and are not limitative to the present invention.

### EXAMPLES

Six solid water-in-oil emulsions for the lips were prepared having the respective compositions given in Tables 1 to 6 hereinbelow (compositions 1 to 6).

The percentages are percentages by weight.

1) Composition 1

TABLE 1

| | | Quantity | |
|---|---|---|---|
| | Trade | (%) (Total = | |
| INCI Name | Name | 100%) | Function |
| Dimethicone | KF-96A- | 35 | Non-volatile |
| | 10CS | | silicon oil |
| Synthetic wax & cera carnauba | Smart wax | 5 | Wax |
| (copernicia cerifera (carnauba) | 202 | | |
| wax) | | | |
| Jojoba esters & polyglycerin-3 | Acticire | 4.5 | Wax |
| & acacia decurrens flower wax | | | |
| & helianthus annuus cera seed | | | |
| (helianthus annuus (sunflower) | | | |
| seed wax) | | | |
| Lauryl PEG-9 polydimeth- | KF-6038 | 2.5 | Silicone |
| ylsiloxyethyl dimethicone | | | emulsifying |
| | | | agent |
| Dimethicone & dimethicone/ | KSG-210 | 2 | Emulsifying |
| PEG-10/15 crosspolymer & | | | silicone |
| dipropylene glycol & | | | elastomer |
| tocopherol* | | | |
| Pigment = blend of iron oxide | — | 2 | Pigment |
| and titanium dioxide | | 4 | ground and |
| Diisostearyl malate | | | dispersed in |
| | | | ester oil |
| | | 3.8 | Pearlescent |
| | | | agents |
| Aqua (water) | Deionised | 40 | Water |
| | water | | |
| Polysorbate 60 | Tween | 0.5 | Non-silicone |
| | 60-LQ | | emulsifying |
| | | | agent |
| Phenoxyethanol | Sepicide | 0.7 | Preservative |
| | LD | | |

*KSG-210 silicone elastomer emulsifier having the INCI name “Dimethicone & Dimethicone/PEG-10/15 Crosspolymer & Dipropylene glycol & tocopherol” is broken down as follows: 74.5% Dimethicone; 25% Dimethicone/PEG-10/15 Crosspolymer; 0.5% Dipropylene glycol; 0.02% tocopherol.

So, 2% of KSG210 breaks down as follows: 1.5% of dimethicone; 0.5% Dimethicone/PEG-10/15 Crosspolymer; 0.01% Dipropylene glycol; 0.0004% tocopherol.

The amount of “active ingredient” emulsifying silicone elastomer in 2% KSG-210 is 0.5%.

Note:

Adding the dispersing agent diisostearyl malate (which is also an ester oil) improves the dispersion of the pigments in the composition.

The procedure implemented for the preparation of composition 1 (Table 1) hereinbefore is as follows.

1) The fatty phase is prepared in a water bath at 95° C. More particularly, the silicone elastomer emulsifier KSG-210 is premixed in the silicone emulsifier KF-6038. The ground pigments (dispersed in malate diisosterayl) and non-volatile silicone oil (KF-96A-10CS) are added. The mixture was stirred manually.

2) The waxes are added to the mixture obtained in 1). The mixture is heated to a temperature between 90° C. and 100° C. and is kept at this temperature for 10 minutes.

3) The mixture obtained in 2) is stirred in a Rayneri mixer at 300 rpm and the pearlescent agents are added.

4) At the same time the aqueous phase is prepared. The non-silicone emulsifier (Tween 60-LQ) is dissolved in deionised water and the preservative is added. The whole is heated in a water bath at a temperature of 90° C.

5) The aqueous phase obtained in 4) is poured, slowly and continuously, into the fatty phase obtained in 3) and vigorously stirred.

6) The mixture obtained in 5) is left emulsified for 5 minutes while keeping the temperature at 90° C.

7) The water-in-oil emulsion thus obtained is hot cast at a temperature between 70° C. and 90° C. in an apparatus suitable for preparing a lipstick.

8) The cast product is allowed to cool before removal from the mould.

2) Composition 2

TABLE 2

| | | Quantity | |
|---|---|---|---|
| | Trade | (%) (Total = | |
| INCI Name | Name | 100%) | Function |
| Dimethicone & | KSG-210 | 2 | Emulsifying |
| Dimethicone/PEG- | | | silicone |
| 10/15 Crosspolymer | | | elastomer |
| & Dipropylene | | | |
| glycol & tocopherol | | | |
| Lauryl PEG-9 | KF-6038 | 2.5 | Silicone |
| polydimeth- | | | emulsifying |
| ylsiloxyethyl | | | agent |
| dimethicone | | | |
| Pigment = blend | — | 2 | Pigment ground |
| of iron oxide | | 4 | and dispersed |
| and titanium dioxide | | | in ester oil |
| Diisostearyl malate | | | |
| Dimethicone | KF-96A- | 28.275 | Non-volatile |
| | 10CS | | silicon oil |
| Ethylhexyl palmitate | Palmitate | 9.425 | Non-volatile |
| | ethyl 2 | | ester oil |
| | hexyl | | |
| Polyethylene | Jeenate 3H | 6 | Wax |
| Polyethylene | Performalene | 1.5 | Wax |
| | 400 | | |
| | | 3.8 | Pearlescent |
| | | | agents |
| Aqua (water) | Deionised | 40 | Water |
| | water | | |
| Polysorbate 60 | Tween | 0.5 | Non-silicone |
| | 60-LQ | | emulsifying |
| | | | agent |

Note:

Non-volatile ester oil used in the composition of the invention, namely 2-ethylhexyl palmitate, improves the compatibility of the hydrocarbon wax with silicone compounds of the formula.

The procedure implemented for the preparation of composition 2 (Table 2) hereinbefore is the same as that of Example 1. 2-ethyl-hexyl palmitate is added in the fatty phase of step 1).

Composition 2 is subsequently poured into a Petri dish and placed for 24 to 48 hours at 20° C. before carrying out the hardness measurement under the conditions described above.

The measurement of the hardness of the composition 2 (Table 2) is 88+/−3 g.

3) Composition 3

TABLE 3

| | | Quantity | |
|---|---|---|---|
| | Trade | (%) (Total = | |
| INCI Name | Name | 100%) | Function |
| Dimethicone & | KSG-210 | 2 | Emulsifying |
| Dimethicone/PEG- | | | silicone |
| 10/15 Crosspolymer | | | elastomer |
| & Dipropylene | | | |
| glycol & tocopherol | | | |
| Lauryl PEG-9 | KF-6038 | 2.5 | Silicone |
| polydimeth- | | | emulsifying |
| ylsiloxyethyl | | | agent |
| dimethicone | | | |
| Pigment = blend | — | 2 | Pigment ground |
| of iron oxide | | 4 | and dispersed |
| and titanium dioxide | | | in ester oil |
| Diisostearyl malate | | | |
| Dimethicone | KF-96A- | 27.775 | Non-volatile |
| | 10CS | | silicon oil |
| Ethylhexyl palmitate | Palmitate | 9.425 | Non-volatile |
| | ethyl 2 | | ester oil |
| | hexyl | | |
| Polyethylene | Jeenate | 6 | Wax |
| | 3H | | |
| Synthetic wax & cera | Smart | 2 | Wax |
| carnauba (copernicia | Wax 202 | | |
| cerifera (carnauba) | | | |
| wax) | | | |
| | | 3.8 | Pearlescent |
| | | | agents |
| Aqua (water) | Deionised | 40 | Water |
| | water | | |
| Polysorbate 60 | Tween | 0.5 | Non-silicone |
| | 60-LQ | | emulsifying |
| | | | agent |

The procedure implemented for the preparation of composition 3 (Table 3) hereinbefore is the same as that described in Example 1.

Composition 3 is subsequently poured into a Petri dish and placed for 24 to 48 hours at 20° C. before carrying out the hardness measurement under the conditions described above.

The hardness of the composition 3 (Table 3) is 80+/−2 g

4) Composition 4

TABLE 4

| | | Quantity | |
|---|---|---|---|
| | Trade | (%) (Total = | |
| INCI Name | Name | 100%) | Function |
| Dimethicone & | KSG-210 | 2 | Emulsifying |
| Dimethicone/PEG- | | | silicone |
| 10/15 Crosspolymer | | | elastomer |
| & Dipropylene | | | |
| glycol & tocopherol | | | |
| Lauryl PEG-9 | KF-6038 | 2.5 | Silicone |
| polydimeth- | | | emulsifying |
| ylsiloxyethyl | | | agent |
| dimethicone | | | |
| Pigment = blend | — | 2 | Pigment ground |
| of iron oxide | | 4 | and dispersed |
| and titanium dioxide | | | in ester oil |
| Diisostearyl malate | | | |
| Dimethicone | KF-96A- | 26.775 | Non-volatile |
| | 10CS | | silicon oil |
| Ethylhexyl palmitate | Palmitate | 9.425 | Non-volatile |
| | ethyl 2 | | ester oil |
| | hexyl | | |
| Polyethylene | Jeenate 3H | 9 | Wax |
| | | 3.8 | Pearlescent |
| | | | agents |
| Aqua (water) | Deionised | 40 | Water |
| | water | | |
| Polysorbate 60 | Tween | 0.5 | Non-silicone |
| | 60-LQ | | emulsifying |
| | | | agent |

The procedure implemented for the preparation of composition 4 (Table 4) hereinbefore is the same as that described in Example 1.

Composition 4 is then poured into a Petri dish and placed for 24 to 48 hours at 20° C. before carrying out the hardness measurement under the conditions described hereinbefore.

The hardness of the composition 4 (Table 4) is 112+/−4 g

5) Composition 5

TABLE 5

| | | Quantity | |
|---|---|---|---|
| | Trade | (%) (Total = | |
| INCI Name | Name | 100%) | Function |
| Dimethicone & | KSG-210 | 2 | Emulsifying |
| Dimethicone/PEG- | | | silicone |
| 10/15 Crosspolymer | | | elastomer |
| & Dipropylene | | | |
| glycol & tocopherol | | | |
| Lauryl PEG-9 | KF-6038 | 2.5 | Silicone |
| polydimeth- | | | emulsifying |
| ylsiloxyethyl | | | agent |
| dimethicone | | | |
| Pigment = blend | — | 2 | Pigment ground |
| of iron oxide | | 4 | and dispersed |
| and titanium dioxide | | | in ester oil |
| Diisostearyl malate | | | |
| Dimethicone | KF-96A- | 27.775 | Non-volatile |
| | 10CS | | silicon oil |
| Ethylhexyl palmitate | Palmitate | 9.425 | Non-volatile |
| | ethyl 2 | | ester oil |
| | hexyl | | |
| Polyethylene | Jeenate 3H | 6 | Wax |
| Jojoba esters | Floraesters | 2 | Wax |
| | 70 | | |
| | | 3.8 | Pearlescent |
| | | | agents |
| Aqua (water) | Deionised | 40 | Water |
| | water | | |
| Polysorbate 60 | Tween | 0.5 | Non-silicone |
| | 60-LQ | | emulsifying |
| | | | agent |

The procedure implemented for the preparation of composition 5 (Table 5) above is the same as that described in Example 1 above, except that the emulsification temperature of step 6 and the casting temperature of step 8 are lowered to a temperature between 70° C. and 80° C. because of the mixture of polyethylene wax “Jeenate 3H” and jojoba esters “Floraester 70”.

Composition 5 is subsequently cast into a small container and placed for 24 to 48 hours at 20° C. before carrying out the hardness measurement under the conditions described hereinbefore.

The hardness of the composition 5 (Table 5) is 86+/−5 g.

6) Composition 6

TABLE 6

| | | Quantity | |
|---|---|---|---|
| | Trade | (%) (Total = | |
| INCI Name | Name | 100%) | Function |
| Dimethicone & | KSG-210 | 2 | Emulsifying |
| Dimethicone/PEG- | | | silicone |
| 10/15 Crosspolymer | | | elastomer |
| & Dipropylene | | | |
| glycol & tocopherol | | | |
| Lauryl PEG-9 | KF-6038 | 2.5 | Silicone |
| polydimeth- | | | emulsifying |
| ylsiloxyethyl | | | agent |
| dimethicone | | | |
| Pigment = blend | — | 5 | Pigment ground |
| of iron oxide | | 7.3 | and dispersed |
| and titanium dioxide | | | in ester oil |
| Diisostearyl malate | | | |
| Dimethicone | XIAMETER | 13.2 | Non-volatile |
| | PMX-200 | | silicon oil |
| | silicone | | |
| | fluide-5CS | | |
| Diisostearyl malate | Salacos | 3 | Non-volatile |
| | 222 | | ester oil |
| Ethylhexyl palmitate | Palmitate | 8 | Non-volatile |
| | ethyl 2 | | ester oil |
| | hexyl | | |
| Polyethylene | Jeenate 3H | 6 | Wax |
| Jojoba esters | Floraesters | 2 | Wax |
| | 70 | | |
| Jojoba esters & | Acticire | 0.1 | Wax |
| polyglycerin-3 & | MB | | |
| Acacia decurrens | | | |
| flower wax & | | | |
| Helianthus annuus | | | |
| cera seed | | | |
| Sodium chloride | | 0.5 | additive |
| Trimeth- | Belsil | 7 | film-forming |
| ylsiloxysilicate | TMS 803 | | agent |
| Shorea Robusta Resin | Kahlresin ® | 1 | film-forming |
| octyldodecanol | 6723 | 1 | agent |
| Aqua (water) | Deionised | 39.2 | Water |
| | water | | |
| phenoxyethanol | | 0.8 | Preservative |
| Anti-oxidant | | 0.7 | Anti-oxidant |
| and perfume | | | and perfume |
| active ingredient | | 0.2 | active |
| | | | ingredient |
| Polysorbate 60 | Tween 60-LQ | 0.5 | Non-silicone |
| | | | emulsifying |
| | | | agent |

The procedure implemented for the preparation of composition 6 (Table 6) hereinbefore is as follows:
1) The film-forming agent is pre-dispersed “Belsil TMS 803” in the non-volatile silicone oil “Xiameter Pmx-Sil 200 Fluid 5CS” to obtain a transparent and homogeneous mixture.
2) The fatty phase is prepared in a water bath at a temperature between 70° C. and 80° C. More preferably the non-volatile oil ester “2 ethyl hexyl palmitate,” waxes “Jeenathe 3H”, “Floraesters 70”, “Acticire MB” and the film-forming agent “Kahlresin® 6723” are mixed via gentle stirring.
3) The emulsifying silicone elastomer “KSG-210” is pre-mixed with the silicone emulsifier “KF-6038”, whereupon this pre-mixture of emulsifiers is added to the mixture of step 2 while maintaining the temperature between 70 and 80° C. and stirring vigorously for 10 minutes at about 3000 rpm (rotation per minute) in an IKA device.
4) The mixture of step 1 is then added to the mixture of step 3 while maintaining the temperature between 70 and 80° C. and stirring vigorously for about 10 minutes at about 3000 rpm.
5) The agitation is reduced and the temperature is maintained between 70° C. and 80° C. for introducing the pigments that were pre-dispersed in malate diisostaryl.
6) In parallel, the water phase is prepared. The non-silicone emulsifier (Tween 60-LQ) is dissolved in deionised water and adding the preservative and the sodium chloride. The whole is heated in a water bath at a temperature between 70° C. and 80° C.
7) The aqueous phase obtained in 6) is poured, slowly and continuously, into the fatty phase obtained in 5) and stirred vigorously.
8) The mixture obtained in 7) is left emulsified for 5 minutes while keeping the temperature between 70° C. and 80° C.
9) The heat sensitive ingredients such as anti-oxidants, active and perfumes are added.
10) The water-in-oil emulsion thus obtained at step 9 is hot cast at a temperature between 70° C. and 80° C. in an apparatus suitable for preparing a lipstick.
11) The cast product is allowed to cool before removal.

Composition 6 is then poured into a sealed box and placed for 24 to 48 hours at 20° C. before carrying out the hardness measurement under the conditions described above.

The hardness of the composition 6 (Table 6) is 100+/−4 g.

Conclusions

The particular choice of silicone emulsifiers, non-volatile silicone oils (and optionally other non-volatile oils, emulsifying silicone elastomers), waxes used in the compositions of the invention provides an especially stable water-in-oil solid emulsion, with a homogeneous appearance and a good hardness (from 70 g to 150 g).

The lipsticks in Tables 1 to 6 hereinbefore (compositions 1 to 6) were applied on the lips of a panel of 9 people.

Each of these lip sticks has excellent sensory properties, namely a feeling of comfort, freshness and melting at the time of application to the skin or lips. They also have a spreadability at the time of application to the skin or lips and a homogeneous result once the composition was applied. Moreover keeping the lipsticks for a period of 3 months at a temperature of 45° C. made it possible to observe that the stick has good stability over time, namely that it does not shrink from its initial shape, it does not break at its base, it does not age by desiccation or drying and it shows no exudation phenomenon (i.e. the release of oil or water on the surface).

In addition, after 3 months at 45° C., these lip sticks maintain excellent sensory properties, namely a feeling of comfort, freshness and melting at the time of application to the skin or lips. They also kept a spreadability at the time of application to the skin or lips and a homogeneous result once the composition was applied.

1-17. (canceled)

18. A cosmetic composition in the form of a solid water-in-oil emulsion comprising:
an emulsifying mixture comprising at least one silicone emulsifier present in an amount between 0.1% and 5% and at least one emulsifying silicone elastomer said silicone elastomer being present in an amount between 0.05% and 3%,
at least one non-volatile oil present in an amount between 30% and 50% and comprising at least one non-volatile silicone oil and optionally a second oil selected from at least one non-volatile ester oil,
a structuring agent of the oily phase comprising a wax and/or an oily gelling agent,
30% to 50% water,
0% to 2%, and still more preferably 0% of a volatile silicone oil and/or a volatile hydrocarbon oil, and
optionally a film-forming agent;
the percentages defined above being percentages by weight relative to the total weight of the composition.

19. The composition according to claim 18, wherein the silicone emulsifier is a siloxane polymer comprising:
a fatty side chain,
an oxyethylenated or oxypropylenated side chain and/or a polyethoxylated side chain, and optionally
a silicone side chain.

20. The composition according to claim 18, wherein the silicone emulsifier is selected from the group comprising:
the compound of formula (I): 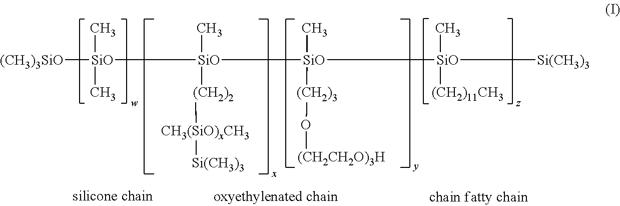
wherein w is an integer from 1 to 1,000, x is an integer between 1 and 50, x, y and z independently of one another represent an integer between 1 and 100,
the compound of formula (II): 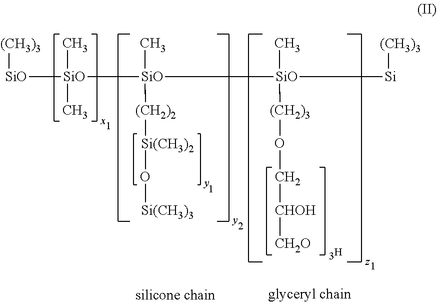
wherein x is an integer between 1 and 1000, w₁ is an integer between 1 and 50, y₁ and z₁ independently of one another represent an integer between 1 and 100,
the compound of formula (III): 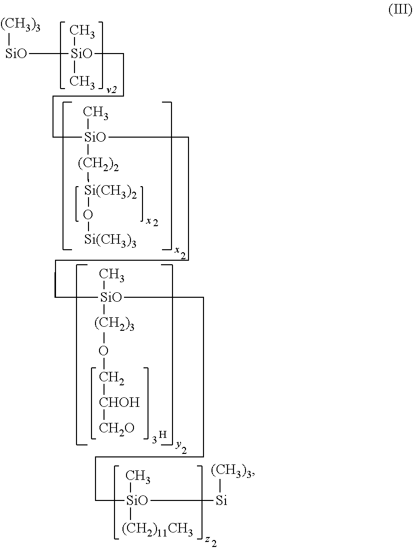
wherein w₂ is an integer between 1 and 1000, v₂ is an integer between 1 and 50, x₂, y₂ and z₂ independently from each other represent an integer between 1 and 100,
and mixtures thereof.

21. The composition according to claim 18, wherein the at least one emulsifying silicone elastomer chosen from crosslinked elastomeric organopolysiloxanes, preferably with a polyhydroxylated chain.

22. The composition according to claim 18, wherein the non-volatile silicone oil is chosen from the group comprising polydimethylsiloxanes (PDMS) containing at least 8 silicon atoms, polyalkylmethylsiloxanes whose alkyl chain contains from 8 to 20 carbon atoms, alkyl dimethicones, vinyl methyl methicones, oils identified by the INCI name phenyltrimethicones and mixtures thereof.

23. The composition according to claim 18, wherein the film-forming agent is selected from silicone resins and especially trimethylsiloxysilicates, acrylates and silicones especially Acrylates/Polytrimethylsiloxymethacrylate Copolymer, polybutenes or plant-derived resins such as Shorea robusta resin or mixtures thereof.

24. The composition according to claim 18, wherein the amount of non-volatile silicone oil is between 10% and 50%, and the amount of film-forming agent when present is between 1% and 15%, preferably between 1% and 10%, the percentages defined above being percentages by weight relative to the total weight of the composition.

25. The cosmetic composition of claim 18, wherein the non-volatile silicone oil has a viscosity at 25° C. greater than or equal to 5 cSt and less than or equal to 10 cSt.

26. The composition according to claim 18, wherein the composition comprises:
a non-volatile ester oil, in particular selected from the group comprising the palmitate, 2-ethylhexyl, isopropyl palmitate, isooctyl palmitate, isocetyl palmitate, isodecyl palmitate isostearyl palmitate, 2-octyl-decyl, isodecyl neopentanoate, isocetyl octanoate, isononyl isononanoate, isodecyl isononanoate, tridecyl isononanoate, hexyl laurate, 2-hexyl-decyl laurate, isopropyl myristate, isocetyl myristate, isotridecyl myristate, 2-octyldodecyl myristate, isopropyl isostearate, octyl-2 dodecyl stearate, isostearyl isostearate, 2-octyldodecyl erucate, diisostearyl malate and mixtures thereof; and optionally
a non-volatile oil selected from the group comprising hydrocarbon oils; (poly) esters and synthetic (poly) ethers, in particular C6-C20 acid (poly) esters and C6-C20 alcohols, preferably branched such as isononyl isononanoate; vegetable oils; branched and/or unsaturated fatty acids; branched fatty alcohols and/or unsaturated such as octyldodecanol; fluorosilicone oils; fluorinated oils; and mixtures thereof.

27. The composition according to claim 18, wherein the structuring agent of the oily phase is:
a polar wax, i.e. a wax comprising at least one heteroatom such as oxygen, nitrogen, silicon or phosphorus,
a hydrocarbon apolar wax, i.e. a wax containing only carbon atoms and hydrogen and containing no hetero atoms such as oxygen, nitrogen, silicon or phosphorus,
a silicone apolar wax, i.e. a wax comprising a silicon heteroatom, and/or,
an oily gelling agent chosen from the group comprising a silicone polymer; a cyclic copolymer of vinyl dimethicone/dimethicone; a polyamide resin or a resin poly (ester-amide); a copolymer of styrene and at least one olefin other than styrene; an N-acyl glutamic acid diamide; a dextrin ester; a sucrose ester and mixtures thereof.

28. The composition according to claim 18, wherein the structuring agent of the oily phase is a wax selected from the group consisting of polyethylene wax, alone or in combination with another polyethylene wax, a mixture of polyethylene wax and of vegetable ester wax, a mixture of synthetic wax and carnauba wax, jojoba esters, and mixtures thereof.

29. The composition according to claim 18, wherein the composition comprises:
an emulsifying mixture comprising at least one silicone emulsifier present in an amount between 0.1% and 5% and at least one emulsifying elastomer silicone said elastomeric silicone being present in an amount between 0.05% and 3%,
at least one non-volatile oil present in an amount between 30% and 50% and comprising at least one non-volatile silicone oil in an amount between 10% and 50% and a second oil selected from at least one non-volatile ester oil,
2% to 12%, preferably 6% to 10%, of a structuring agent for the oily phase,
30% to 50% water,
0% to 2% and preferably 0% of a volatile silicone oil and/or a volatile hydrocarbon oil, and
optionally a film-forming agent in an amount less than or equal to 10%;
the percentages defined hereinbefore being percentages by weight relative to the total weight of the composition.

30. The composition according to claim 18, further comprising:
a non-silicone emulsifier, chosen in particular from the group comprising polymers of the glycol fatty acid ester polyoxyalkylene type, such as those identified by the INCI name Polysorbate 60, Polysorbate 20, Polysorbate 85 and mixtures thereof,
a humectant selected from the group consisting of glycerol; glycols, such as propylene glycol, butylene glycol and hexylene glycol; sugar alcohols or sugar polyols such as sorbitol, xylitol and maltitol; polymer polyols such as polydextrose; alpha-hydroxy acids such as lactic acid; hyaluronic acid; urea; L-pyrrolidone carboxylic acid (PCA); and mixtures thereof,
an additive elected from the group consisting of talc, mica, silica, kaolin, boron nitride, lauroyl lysine, sericite, cellulose beads, glass beads, starch, starch modified by octenyl succinic anhydride, silicone resins such as polymethylsilsesquioxane available under the Momentive Performance Materials tradename Tospearl 2000B, powders derived from silicone elastomers such as silicone elastomer coated with a silicone resin available under the ShinEtsu tradenames KSP 100 or KSP300 of, and mixtures thereof
a colorant selected from the group comprising water-soluble dyes, liposoluble dyes, particles having the effect of colouring and/or opacifying the composition, such as pigments, pearlescent agents, lacquers (water-soluble dyes adsorbed onto a support inert inorganic), and mixtures thereof,
a preservative selected from the group consisting of phenoxyethanol, potassium sorbate, sodium dehydroacetate, chlorphenesin, parabens such as methylparaben or propylparaben, and mixtures thereof and/or
sodium chloride.

31. The composition according to claim 30, wherein the composition comprises:
0% to 5% of a non-silicone emulsifier, preferably 0.1% to 1%,
0% to 10% of a humectant, preferably 1% to 6%,
0% to 25% an additive, preferably 2% to 10%,
0% to 10% of a colorant, preferably 2% to 6%,
0% to 2% of a preservative, preferably 0.1% to 1%,
0% to 1.5% sodium chloride, preferably 0.3 to 0.7%,
the percentages defined above being percentages by weight relative to the total weight of the composition.

32. The composition according to claim 18, wherein the composition has a hardness of between 70 g and 150 g (grams), preferably between 80 g and 120 g, and even more preferably between 80 g and 100 g, said hardness being determined by measuring the compression strength measured at 20° C. using a texturometer marketed by Swantech under the name “TA-XT Plus Microstable System”.

33. The composition according to claim 18, wherein the composition is in the form of a stick, preferably a make-up stick for the skin or the lips and/or non-therapeutic care stick for the skin or lips.

34. A cosmetic method for making up and/or for the nontherapeutic care of the skin or the lips, comprising applying a composition as defined in claim 18 to the skin or the lips.
